(19) United States
(12) Patent Application Publication
(10) Pub. No.: US 2005/0256440 A1
(43) Pub. Date: Nov. 17, 2005

(54) Disposable device for mechanically engaging a vagina

(76) Inventors: MaryAnn Zunker, Oshkosh, WI (US); Ann Marie Przepasniak, Appleton, WI (US); Cornelis J. Bosselaar, Appleton, WI (US)

Correspondence Address: SCHWEGMAN, LUNDBERG, WOESSNER & KLUTH, P.A., P.O. BOX 2938, MINNEAPOLIS, MN, 55402-0938, US

(21) Appl. No.: 10/836,102

(22) Filed: Apr. 30, 2004

(51) Int. Cl. A61B 10/00; A61M 35/00

(52) U.S. Cl. 604/1

(57) ABSTRACT

In some embodiments, the present invention relates to a disposable device for mechanically engaging a vagina. The disposable device includes an elongated handle and an elongated swab that is secured to the elongated handle. The elongated swab has an oblong cross-section in a plane that is orthogonal to a longitudinal axis of the elongated swab. In other embodiments, the present invention relates to a method of fabricating a disposable device for mechanically engaging a vagina. The method includes forming an elongated swab that has an oblong cross-section in a plane which is orthogonal to a longitudinal axis of the elongated swab. The method further includes securing the elongated swab to an elongated handle. In other embodiments, the present invention relates to a method of engaging a vagina. The method includes positioning the disposable device near a vagina and mechanically maneuvering the elongated handle to manipulate the elongated swab against the vagina.

### FIELD OF THE INVENTION

Some embodiments of the present invention relate to a device for engaging a vagina, and in particular to a disposable device for mechanically engaging a vagina.

### BACKGROUND OF THE INVENTION

Normal vaginal secretions are clean, mucoid, and have high lubricating properties such that there is always a natural moistness to a vagina. There is also typically some debris present in the vagina. The debris is usually in the form of desquamating epithelial cells. In addition, blood left from menstrual flow and/or deposited seminal fluid also often add to the debris found in the vagina.

Since the temperature of a vagina is constantly maintained at the body temperature of a woman, the vagina makes an excellent incubator for a long list of protozoa, fungi and bacteria (both pathogenic and nonpathogenic). In addition, the largely protein nature of vaginal debris, as well as the presence of carbohydrates (e.g., glycogen), further promote the formation of protozoa, fungi and bacteria within the vagina.

A typical vagina normally has an acidity that inhibits the propagation of most harmful pathogens to some degree. However, this acidity does not kill the pathogens but merely restrains their multiplication. The harmful pathogens are always present and ready to grow, especially when the environment within the vagina undesirably changes to allow pathogen growth.

Accordingly, vaginal cleansing should be a regular part of personal hygiene. Most gynecologists recommend douches of some kind when vaginal discharge causes the patient to complain of discomfort or embarrassment due to odor, itching and irritation (among other symptoms).

A douche is typically in the form of a fluid that is introduced into the vagina. One of the drawbacks with conventional vaginal douches is that they tend to remove both good and bad flora in the vagina. In addition, using conventional douches often leaves the mucosal tissue within the vagina dry and irritated. Another drawback associated with using douches is that when the cleansing fluid is inserted into the vagina, the fluid can push bacteria into, and through, the cervix leading to infections.

There is a need for a disposable device that mechanically engages a vagina. The device should clean the vagina without leaving the vagina dry and irritated and without using free flowing fluid. The device should also be able to insert materials into the vagina. It would also be desirable if the device were small portable, discreet and sanitary. There is also a need to minimize the cost associated with producing the device.

### SUMMARY OF THE INVENTION

In response to the drawbacks described above with respect to conventional vaginal douches, a new disposable device for mechanically engaging a vagina has been invented.

One aspect of the invention relates a disposable device for mechanically engaging a vagina. The disposable device includes an elongated handle having a first end and a second end and an elongated swab that is secured to the elongated handle. The elongated swab has an oblong cross-section in a plane that is orthogonal to a longitudinal axis of the elongated swab. In some embodiments, the oblong cross-section has an elliptical shape with a width between 30 mm and 50 mm and a thickness between 2 mm and 30 mm. In addition, the elongated swab may be tapered anywhere from the first end to the second end of the elongated swab. The elongated handle may also have an oblong cross-section in a plane that is orthogonal to the longitudinal axis of the elongated handle and/or may include a spoon-shaped section to facilitate grasping the handle.

Another aspect of the invention relates to a method of fabricating a disposable device for mechanically engaging a vagina. The method includes forming an elongated swab that includes a first end, a second end and a longitudinal axis where the elongated swab has an oblong cross-section in a plane which is orthogonal to the longitudinal axis. The method further includes securing the elongated swab to an elongated handle. In some embodiments, securing the elongated swab to the elongated handle includes embedding the elongated handle in the elongated swab. The method may also include forming the elongated swab with a cleanser or other active ingredient (e.g., a spermicide).

Still another aspect of the invention relates to a method of engaging a vagina. The method includes positioning a disposable device for mechanically engaging a vagina near a vagina where the disposable device includes an elongated handle and an elongated swab that is secured to the elongated handle. The elongated swab has an oblong cross-section in a plane that is orthogonal to a longitudinal axis of the elongated swab. The method further includes mechanically maneuvering the elongated handle to manipulate the elongated swab against the vagina. Positioning a disposable device near a vagina includes positioning the disposable device within the vagina, and mechanically maneuvering the elongated handle includes mechanically maneuvering the elongated handle outside the vagina (e.g., by rotating the handle) to manipulate the elongated swab inside the vagina. In some embodiments, mechanically maneuvering the elongated handle includes mechanically maneuvering the elongated handle to clean the vagina and/or to apply a material to the vagina.

### BRIEF DESCRIPTION OF THE DRAWINGS

The above-mentioned and other features will become more apparent and better understood by reference to the following description of the invention taken in conjunction with the accompanying drawings.

FIG. 1 is a side view illustrating an example disposable device for mechanically engaging a vagina of the present invention.

FIG. 2 is a section view of the elongated swab that is used in disposable device shown in FIG. 1 taken along line 2-2 in FIG. 1.

FIG. 3 is a section view similar to FIG. 2 illustrating a cross-section of another example elongated swab that may be used in the disposable device shown in FIG. 1.

FIG. 4 is a side view illustrating another example disposable device for mechanically engaging a vagina of the present invention.

FIG. 5 is a side view illustrating another example disposable device for mechanically engaging a vagina of the present invention.

FIG. 6 is a side view illustrating another example disposable device for mechanically engaging a vagina of the present invention.

FIG. 7 is a section view of the elongated handle that is used in the disposable device shown in FIG. 1 taken along line 7-7 in FIG. 1.

FIG. 8 is a side view illustrating another example disposable device for mechanically engaging a vagina of the present invention.

FIG. 9 is a schematic view illustrating a method of engaging a vagina according to the present invention.

### DESCRIPTION OF THE INVENTION

The disposable device for mechanically engaging a vagina of the present invention may provide an efficient, healthy and low-cost way to clean inside a vagina without using a douching product. The disposable device may also provide an effective way to apply a material to a vagina.

As used herein, “disposable device” includes a device that is disposed of after use and is not intended for reuse. The disposable device described herein may be especially useful to an individual that needs to mechanically clean (or apply materials to) their vagina in a variety of situations.

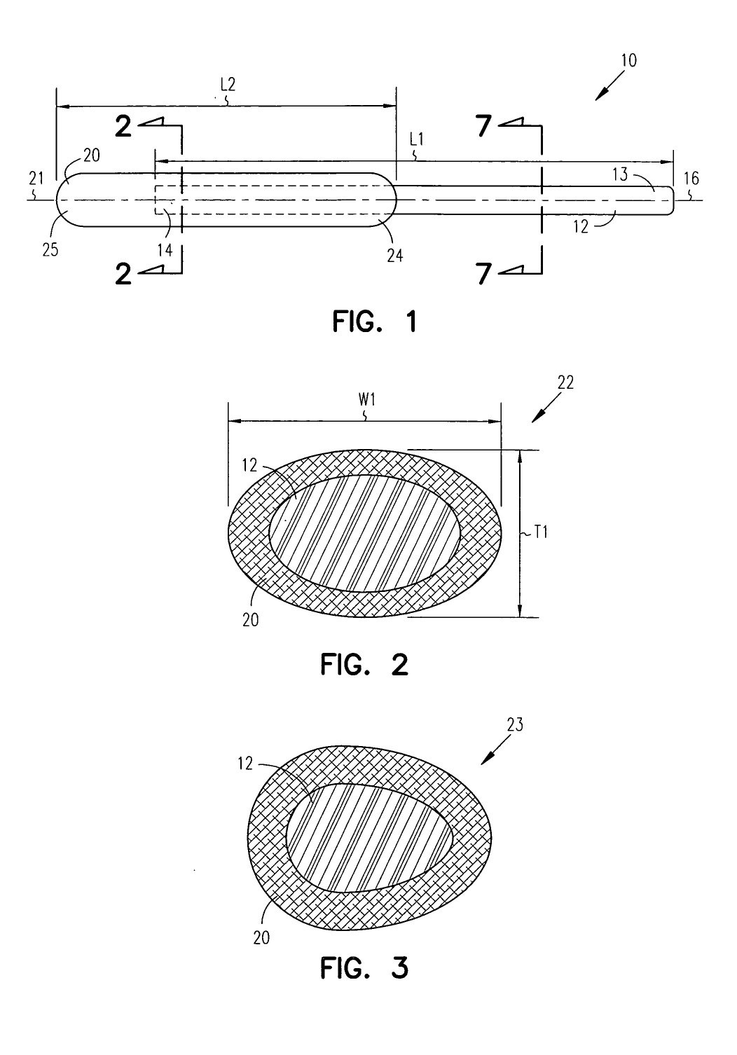

FIG. 1 illustrates an example disposable device 10 for mechanically engaging a vagina of the present invention. The disposable device 10 includes an elongated handle 12 having a first end 13 and a second end 14. The disposable device 10 further includes an elongated swab 20 that has a longitudinal axis 21. The elongated swab 20 is secured to the second end 14 of the elongated handle 12.

The elongated swab 20 has an oblong cross-section in a plane that is orthogonal to the longitudinal axis 21 of the elongated swab 20. As used herein, “oblong” means elongated from a circular form in one or more directions (see, e.g., elliptical cross-section 22 in FIG. 2 and egg shaped cross-section 23 in FIG. 3).

The elongated swab 20 may be constructed of any material that is suitable for engaging a vagina. Some example materials include cotton, rayon, wood pulp and polymeric substances such as nonwoven fabrics, foam sponges and thermoplastics.

In addition, the elongated swab 20 may be designed with any type of surface that enhances the cleansing and/or insertion properties of the disposable device 10. In some embodiments, the surface of the elongated swab 20 may be textured to facilitate removal of residue and film from the vagina.

In the example embodiment illustrated in FIG. 2, the oblong cross-section 22 has a thickness T1 and a width W1. As an example, the width W1 of the oblong cross-section 22 may be between 30 mm and 50 mm, and the thickness T1 may be between 2 mm and 30 mm. In some embodiments, the width W1 of the oblong cross-section may be at least twice as large as the thickness T1.

In some embodiments, the elongated swab 20 may be composed of a nonwoven fabric. As used herein, “nonwoven fabric” refers to a web or fabric (made partly or wholly of non-cellulose material) that includes individual fibers which are interlaid (randomly or in a pattern) in a knitted fabric. The nonwoven webs may be formed by many processes, including meltblowing, spunbonding and bonded carded. The nonwoven fabrics that are used in the elongated swab 20 may be produced from polymers such as polyethylene or polypropylene.

As discussed above, the elongated swab 20 may include a textured surface. Some example nonwoven textured materials include rush transfer materials, flocked materials, wireform nonwovens and thermal point unbonded materials (among others).

In some embodiments, the elongated swab 20 may be formed of a nonwoven fabric that is made of an array of interbonded thermoplastic fibers which may be formed from a wide variety of thermoplastic materials. Some example thermoplastic materials include polyolefins (e.g., polyethylene, polypropylene), polystyrene and polyamides (e.g., nylon). In addition, thermoplastic polymers that are elastomeric may also be used as fibers. Some example elastomeric materials include polyurethanes and block copolymers. It should be noted that blends of any of the foregoing materials may be used to form the fibers.

The fibers may also include additives (e.g., wax, pigments, stabilizers and fillers) that are inserted into the fibers as the fibers are fabricated to achieve one or more desired properties within the fibers. Some example additives include compatible surfactants that may be added to the polymers to make the surface of the fibers more wettable and thereby improve the ability of the disposable device 10 to attract unwanted debris within the vagina. It should be noted that the amount of surfactant which may be added to the fibers can be adjusted to control the surface wetting of the fabric that is formed from the fibers. Some example surfactants that may be added to the fibers include sodium dioctyl sulfosuccinate and alkyl phenoxy ethanol (among others).

In some embodiments, the elongated swab 20 may include a cover material that is placed on the elongated swab 20 to prevent fiber sloughing. In addition, some cover materials may improve the wettablilty of the elongated swab 20. There also some cover materials that may provide stain-masking. In addition, some cover materials may be capable of capturing and/or storing material within the cover material itself, or the trapping the material within the inner portion of the elongated swab 20.

As examples, the cover material may store and/or capture debris, cleansers, lubricants, spermicidal agents and/or medications (among other materials) before and/or after use of the disposable device 10. Some example cover materials include spunbound, spunlace, bonded carded web and apertured film materials. In one example embodiment, the cover material is an apertured film that formed of a polyolefin which may or may not be combined with a nonwoven fabric.

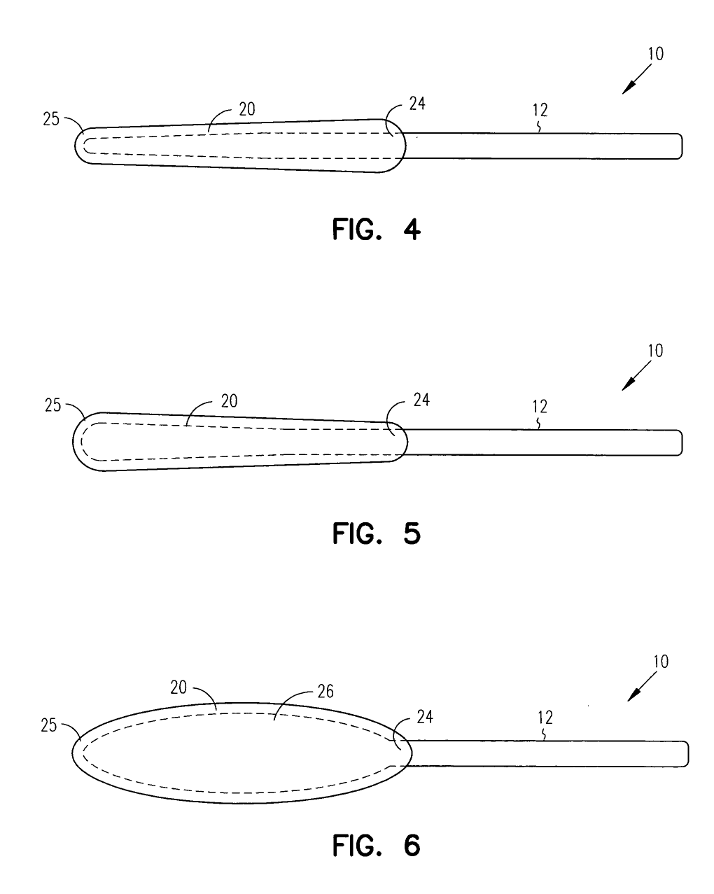

FIG. 4 illustrates an example embodiment where the elongated swab 20 is tapered from a first end 24 to a second end 25 such that the oblong cross-section is larger at the first end 24 than the second end 25. As an example, the width of the oblong cross-section at the first end 24 of the elongated swab 20 may be between 30 mm and 50 mm, and the thickness may be between 2 mm and 30 mm. In addition, the width of the oblong cross-section at the second end 25 may be between 3 mm and 10 mm, and the thickness may be between 2 mm and 5 mm.

FIG. 5 illustrates an example embodiment of the disposable device 10 where the elongated swab 20 is tapered from the first end 24 to the second end 25 such that the oblong cross-section is larger at the second end 25 than the first end 24. As an example, the width of the oblong cross-section at the first end 24 may be between 3 mm and 10 mm, and the thickness may be between 2 mm and 5 mm. In addition, the width of the oblong cross-section at the second end 25 may be between 30 mm and 50 mm, and the thickness may be between 2 mm and 30 mm.

Another example embodiment is shown in FIG. 6 where the elongated swab 20 is tapered from a midsection 26 of the elongated swab 20 to the first and second ends 24, 25. Therefore, the oblong cross-section is larger at the midpoint 26 of the elongated swab 20 than at the first and second ends 24, 25. As an example, the width of the oblong cross-section at the midsection 26 of the elongated swab 20 may be between 30 mm and 50 mm, and the thickness may be between 2 mm and 30 mm. In addition, the width of the oblong cross-section at the first and second ends 24, 25 of the elongated swab 20 may be between 3 mm and 10 mm, and the thickness may be between 2 mm and 5 mm.

When a nonwoven fabric is used as the material for the elongated swab 20, the basis weight of the nonwoven fabric may vary depending on the properties that are desired within the elongated swab 20. As an example, the basis weight for the nonwoven fabric may be as low as 10 grams per square meter and as high as 300 grams per square meter.

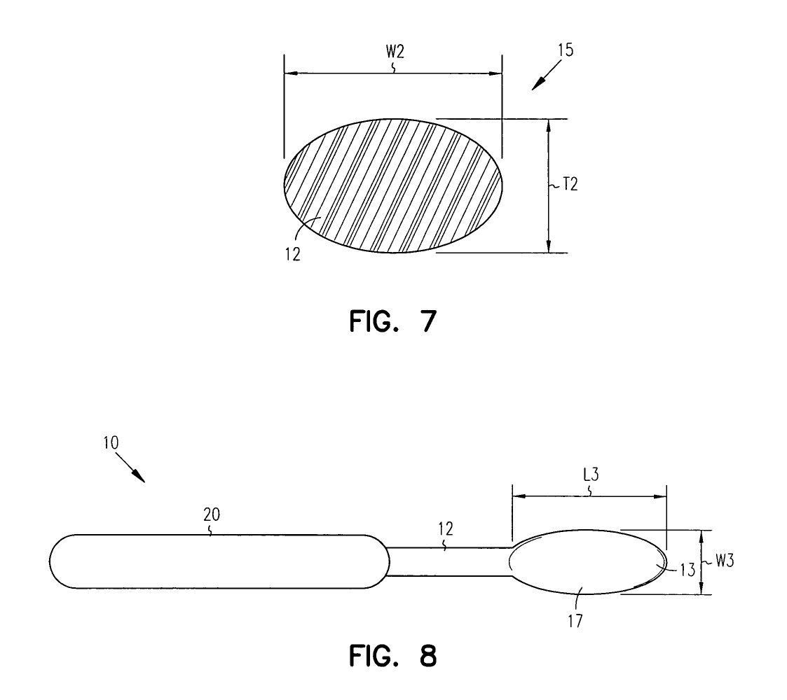

In the example embodiment illustrated in FIGS. 1 and 7, the elongated handle 12 has an oblong cross-section 15 in a plane that is orthogonal to a longitudinal axis 16 of the elongated handle 12. The cross-section of the elongated handle 12 may take other forms (e.g., circle, square, rectangle and polygon) depending on the properties that are desired within the disposable device 10. In other embodiments, the elongated handle 12 may be designed with a size and/or shape that improve the cleansing and/or insertion effectiveness of the disposable device 10.

There are some embodiments where the elongated handle 12 includes one or more sections that make it easier to grasp the disposable device 10. As an example, FIG. 8 shows the disposable device 10 as including a spoon-shaped section 17 at the first end 13 of the elongated handle 12.

The elongated handle 12 may be made from any material that is suitable for insertion into a vagina. The elongated handle 12 should be made from a material that is non-toxic, low cost and hygienic. Some example materials that may be used for the elongated handle 12 include paper, cardboard, polyethylene and polypropylene (among other materials).

Referring again to FIG. 1, the elongated handle 12 has a first length L1 and the elongated swab 20 has a second length L2. As an example, the first length L1 of the elongated handle 12 may be between 40 mm and 250 mm, and the second length L2 the elongated swab 20 may be between 50 mm and 150 mm. In some embodiments, the second length L2 may be longer than the first length L1 of the elongated handle 12. FIG. 1 also illustrates that the second end 14 of the elongated handle 12 may be embedded in the elongated swab 20.

The relative lengths L1, L2 of the elongated handle 12 and the elongated swab 20 may vary depending on the desired properties of the disposable device. In some embodiments, the elongated handle 12 and the elongated swab 20 may be sized to give the elongated swab 20 engagement with the interior of the vagina while keeping elongated handle 12 far enough outside the vagina to allow a user to adequately manipulate the disposable device 10. In addition, the size of the elongated handle 12 and the elongated swab 20 may be short enough to allow a user to generate sufficient pressure on the vagina with the elongated swab 20.

The elongated handle 12 may be solid or hollow. When the elongated handle 12 is hollow, the wall the elongated handle 12 should be thick enough to provide sufficient rigidity to the disposable device 10 during vaginal insertion.

The width of the elongated handle 12 may vary depending on functional properties that are desired of the disposable device 10. In some embodiments, the elongated handle may be small enough to make it easy for a user to insert the disposable device into the vagina while being wide enough to give the elongated handle 12 sufficient rigidity to properly clean the vagina. As an example, the width W2 of the oblong cross-section 15 that is shown in FIG. 7 may be between 5 mm and 40 mm, and the thickness T2 may be between 2 mm and 30 mm. In another example embodiment, the spoon-shaped section 17 shown in FIG. 8 may have a width W3 between 10 mm and 20 mm and a length L3 between 20 mm and 40 mm. In addition, the spoon-shaped section 17 may have a thickness between 1 mm and 5 mm. It should be noted the spoon-shaped section 17 may be flat or include any combination of convex and concave surfaces.

As shown in FIGS. 4-6, the elongated handle 12 may change shape at the portion of the elongated handle 12 which is embedded in the elongated swab 20. In addition, the elongated swab 20 may have a uniform thickness around the elongated handle 12 at the portion of the elongated swab 20 where the elongated handle 12 is embedded in the elongated swab 20.

In some embodiments, a cleanser (not shown) may be applied to the elongated swab 20. The term “cleanser” refers to any paste, gel, powder, oil, liquid or any other appropriate medium that may be used in vaginal cleansing. Some example cleansers include surfactants such as water soluble polymers, polysorbates, glycerins, glycol-based surfactants and/or silicone-based surfactants. It should be noted that the elongated swab may also include other materials, such as water, vinegars, salts, humectants, scouring powders, thickening agents and aromas (among others).

The cleanser may be applied to the elongated swab 20 such that the cleanser is moist to the touch, or the cleanser may be dry and then activated upon contact with the vaginal tissue. The cleanser should not kill significant amounts of lactobacillus while removing unwanted debris during mechanical cleansing of the vagina. In addition, the cleanser should not contain ingredients that might lead to dry vaginal mucosal tissue. In some embodiments, the cleanser may include a moisturizer that helps to maintain a normal hydration level after mechanical cleansing. The cleanser may also contain preservatives and other ingredients that do not disrupt the normal flora of the vaginal vault (e.g., sorbic acid, citric acid, methyl paraben and natural preservatives such as grapefruit extract).

The cleanser may be applied to the elongated swab 20 by various means including spray-coating, dipping, doning, impregnating, electro-spinning coating and inkjet printing (among others). In addition, the cleanser may be applied to the elongated swab 20 at any point in time. As examples, the cleanser may be applied to the elongated swab 20 just prior to using the disposable device 10 to mechanically clean a vagina, or the cleanser may be “pre-applied” to the elongated swab 20. As used herein, “pre-applied” refers to applying the cleanser to the elongated swab 20 during a manufacturing process of the disposable device 10 and/or the elongated swab 20. In addition, “pre-applied” also refers to applying the cleanser to the elongated swab 20 before the sale of disposable device 10.

It should be noted that other materials besides cleansers may be applied to the elongated swab 20 such that the disposable device 10 can be used to apply materials to the vagina. Although any appropriate material may be added to the elongated swab 20, some example materials include lubricants and other inactive or active ingredients (e.g., a spermicidal agent or a medication). Another example material may be a motility enabler that facilitates sperm motility. Any of the materials that may be applied to the elongated swab 20 may be in any form (e.g., liquid, powder and paste).

A method of fabricating a disposable device 10 for mechanically engaging a vagina will now be described with reference to FIG. 1. The method includes forming an elongated swab 20 that includes a first end 24, a second end 25 and a longitudinal axis 21 such that the elongated swab 20 has an oblong cross-section (see, e.g., cross-section 22 in FIG. 2 and cross-section 23 in FIG. 3) in a plane that is orthogonal to the longitudinal axis 21 of the elongated swab 20. The method further includes securing the elongated swab 20 to an elongated handle 12. As used herein, the term “securing” means permanently bonding. Any securing, or bonding, of the elongated handle 12 to the elongated swab 20 may occur at any time during the fabrication of the disposable device 10. Some of the example methods that may be used to bond the elongated swab 20 to the elongated handle 12 include adhesive bonding, thermal bonding and ultrasonic bonding (among others).

The method may further include forming the elongated handle 12 such that the elongated handle 12 includes a first end 13, a second end 14 and a longitudinal axis 16. In some embodiments, forming the elongated handle 12 may include (i) forming the elongated handle 12 to have an oblong cross-section 15 in a plane that is orthogonal to the longitudinal axis 16 of the elongated handle 12 (see FIGS. 1 and 7); (ii) forming the elongated handle 12 to have a spoon-shaped section 17 at the first end 13 of the elongated handle 12 (see FIG. 8); and/or (iii) forming the elongated swab 20 with another material (e.g., a cleanser) that is applied to the elongated swab 20.

Securing the elongated swab 20 to the elongated handle 12 may include embedding the elongated handle 12 in the elongated swab 20 such that the overall length of the disposable device 10 may be between 90 mm and 260 mm. In some embodiments, forming the elongated handle 12 may include forming the elongated handle 12 such that the elongated handle 12 changes shape at a portion of the elongated handle 12 which is embedded in the elongated swab 20. In addition, forming the elongated swab 20 may include forming the elongated swab 20 to have a uniform thickness around the portion of the elongated handle 12 that is embedded in the elongated swab 20.

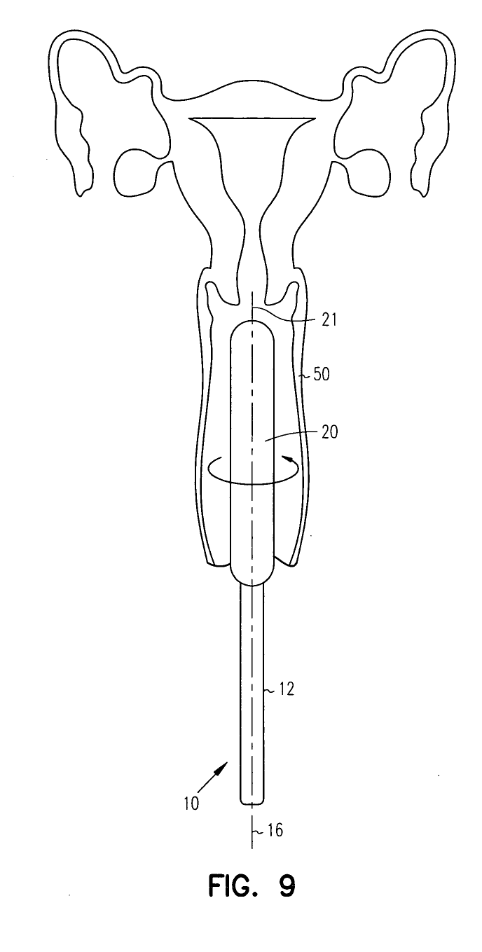

A method of engaging a vagina will now be described with reference to FIG. 9. The method includes positioning a disposable device 10 near a vagina 50. The disposable device 10 includes an elongated handle 12 and an elongated swab 20 that is secured to the elongated handle 12. The elongated swab 20 has an oblong cross-section (see, e.g., cross-section 22 in FIG. 2 and cross-section 23 in FIG. 3) in a plane that is orthogonal to a longitudinal axis 21 of the elongated swab 20. The method further includes mechanically maneuvering the elongated handle 12 to manipulate the elongated swab 20 against the vagina 50.

In some embodiments, positioning a disposable device 10 near a vagina 50 may include positioning the disposable device 10 within the vagina 50. In addition, mechanically maneuvering the elongated handle 12 may include mechanically maneuvering the elongated handle 12 outside the vagina 50 (e.g., by rotating the elongated handle 12) to manipulate the elongated swab 20 inside the vagina 50.

The disposable device 10 that is used in the method shown in FIG. 9 is also the disposable device 10 shown in FIG. 1. Therefore, mechanically maneuvering the elongated handle 12 may include grasping an elongated handle 12 that has an oblong cross-section (see cross-section 15 in FIG. 7) in a plane which is orthogonal to a longitudinal axis 16 of the elongated handle 12. In some embodiments, mechanically maneuvering the elongated handle 12 may include cleaning the vagina and/or applying a material to the vagina.

Any of the disposable devices 10 shown and/or described herein may used in the method of engaging a vagina. As an example, the disposable device 10 shown in FIG. 8 may be used in the method of engaging a vagina such that mechanically maneuvering the elongated handle 12 includes grasping the spoon-shaped section 17 at the first end 13 of the elongated handle 12.

While the invention has been described in detail with respect to specific embodiments, it will be appreciated that there are variations of, and equivalents to these embodiments. Accordingly, the scope of the present invention should be determined by the appended claims and any equivalents thereto.

1. A disposable device for mechanically engaging a vagina, the device comprising:
an elongated handle that includes a first end and a second end; and
an elongated swab that is secured to the elongated handle, the elongated swab having an oblong cross-section in a plane that is orthogonal to a longitudinal axis of the elongated swab.

2. The disposable device of claim 1 wherein the oblong cross-section has an elliptical shape.

3. The disposable device of claim 1 wherein the oblong cross-section has an egg shape.

4. The disposable device of claim 1 wherein the oblong cross-section has a thickness and a width, the width of the oblong cross-section being at least 1.5 times as large as the thickness.

5. The disposable device of claim 1 wherein the oblong cross-section has a thickness and width, the width of the oblong cross-section being between 30 mm and 50 mm and the thickness between 2 mm and 30 mm.

6. The disposable device of claim 1 wherein the elongated swab includes a first end and a second end, the first end of the elongated swab being secured to the elongated handle, the elongated swab being tapered from the first end of the elongated swab to the second end of the elongated swab such that the oblong cross-section is larger at the first end of the elongated swab than the second end of the elongated swab.

7. The disposable device of claim 1 wherein the elongated swab includes a first end and a second end, the first end of the elongated swab being secured to the elongated handle, the elongated swab being tapered from the first end of the elongated swab to the second end of the elongated swab such that the oblong cross-section is larger at the second end of the elongated swab than the first end of the elongated swab.

8. The disposable device of claim 1 wherein the elongated swab includes a first end and a second end, the first end of the elongated swab being secured to the elongated handle, the elongated swab being tapered from a midsection of the elongated swab to the first and second ends of the elongated swab such that the oblong cross-section is larger at the midpoint of the elongated swab than the first and second ends of the elongated swab.

9. The disposable device of claim 1 wherein the elongated handle includes a longitudinal axis, the elongated handle having an oblong cross-section in a plane that is orthogonal to the longitudinal axis of the elongated handle.

10. The disposable device of claim 1 wherein the elongated handle includes a spoon-shaped section.

11. The disposable device of claim 10 wherein the spoon-shaped section of the elongated handle is at a first end of the elongated handle.

12. The disposable device of claim 1 wherein the elongated handle has a first length and the elongated swab has a second length that is larger than the first length of the elongated handle.

13. The disposable device of claim 1 wherein the elongated handle has a first length that is between 40 mm and 250 mm and the elongated swab has a second length that is between 50 mm and 150 mm.

14. The disposable device of claim 1 wherein the elongated handle is embedded in the elongated swab.

15. The disposable device of claim 14 wherein the elongated handle changes shape at a portion of the elongated handle which is embedded in the elongated swab.

16. The disposable device of claim 15 wherein the elongated swab has a uniform thickness around the elongated handle at the portion of the elongated swab where the elongated handle is embedded in the elongated swab.

17. The disposable device of claim 1 wherein the elongated swab includes a cleanser.

18. The disposable device of claim 1 wherein the elongated swab includes a spermicide.

19. A method of fabricating a disposable device for mechanically engaging a vagina, the method comprising:
forming an elongated swab that includes a first end, a second end and a longitudinal axis, the elongated swab having an oblong cross-section in a plane that is orthogonal to the longitudinal axis of the elongated swab; and
securing the elongated swab to an elongated handle.

20. The method of claim 19 further comprising forming the elongated handle to include a first end, a second end and a longitudinal axis.

21. The method of claim 19 wherein forming the elongated handle includes forming the elongated handle to have an oblong cross-section in a plane that is orthogonal to the longitudinal axis of the elongated handle.

22. The method of claim 20 wherein forming the elongated handle includes forming the elongated handle to have a spoon-shaped section.

23. The method of claim 19 wherein securing the elongated swab to the elongated handle includes embedding the elongated handle in the elongated swab.

24. The method of claim 23 wherein forming the elongated handle includes forming the elongated handle such that the elongated handle changes shape at a portion of the elongated handle which is embedded in the elongated swab.

25. The method of claim 24 wherein forming the elongated swab includes forming the elongated swab to have a uniform thickness around the portion of the elongated handle that is embedded in the elongated swab.

26. The method of claim 19 wherein forming the elongated swab includes forming the elongated swab with another material applied to the elongated swab.

27. The method of claim 26 wherein forming the elongated swab with another material applied to the elongated swab includes forming the elongated swab with a cleanser.

28. A method of engaging a vagina, the method comprising:
positioning a disposable device for mechanically engaging a vagina near a vagina, the disposable device including an elongated handle and an elongated swab that is secured to the elongated handle, the elongated swab having an oblong cross-section in a plane that is orthogonal to a longitudinal axis of the elongated swab; and
mechanically maneuvering the elongated handle to manipulate the elongated swab against the vagina.

29. The method of claim 28 wherein positioning a disposable device near a vagina includes positioning the disposable device within the vagina, and mechanically maneuvering the elongated handle includes mechanically maneuvering the elongated handle outside the vagina to manipulate the elongated swab inside the vagina.

30. The method of claim 28 wherein mechanically maneuvering the elongated handle includes grasping a spoon-shaped section of the elongated handle.

31. The method of claim 28 wherein mechanically maneuvering the elongated handle includes grasping an elongated handle that includes a longitudinal axis, the elongated handle having an oblong cross-section in a plane that is orthogonal to the longitudinal axis of the elongated handle.

32. The method of claim 28 wherein mechanically maneuvering the elongated handle includes rotating the elongated handle.

33. The method of claim 28 wherein mechanically maneuvering the elongated handle includes mechanically maneuvering the elongated handle to clean the vagina.

34. The method of claim 28 wherein mechanically maneuvering the elongated handle includes mechanically maneuvering the elongated handle to apply a material to the vagina.
